(19) United States
(12) Patent Application Publication
(10) Pub. No.: US 2006/0013844 A1
(43) Pub. Date: Jan. 19, 2006

(54) Facial cosmetic devices and systems

(76) Inventor: Cathy Meriaux, Billerica, MA (US)

Correspondence Address: LAW OFFICES OF ERIC KARICH, 2807 ST. MARK DR., MANSFIELD, TX, 76063, US

(21) Appl. No.: 10/998,203

(22) Filed: Nov. 26, 2004

Related U.S. Application Data
(60) Provisional application No. 60/587,649, filed on Jul. 14, 2004.

(51) Int. Cl. A61K 8/02

(52) U.S. Cl. 424/402

(57) ABSTRACT

Several embodiments of a cosmetic device are disclosed. In general, the cosmetic device includes a thin, flexible substrate having two opposed sides, and an adhesive layer formed on one of the sides of the substrate. In some embodiments, a coloring layer is formed on the other side of the substrate. In another embodiment, multiple hair elements are distributed across, and have portions extending outwardly from, the other side of the substrate. Described cosmetic systems include multiple cosmetic devices releasably attached to release sheets.

### CROSS-REFERENCE TO RELATED APPLICATIONS

This application for a utility patent claims the benefit of U.S. Provisional Application No. 60/587,649, filed Jul. 14, 2004. This application is incorporated herein by reference in its entirety.

### BACKGROUND OF THE INVENTION

1. Field of the Invention

This invention relates generally to cosmetic devices, and more particularly to a cosmetic device that is applied to the face in sheet form with an adhesive.

2. Description of Related Art

In general, the term “cosmetic” refers to something applied to the body (e.g., the skin or hair) to beautify or to improve a deficiency, defect, or irregularity. The term “makeup” generally refers to cosmetics applied to the face.

It's human nature to want to be considered physically attractive. Evidence of the use of eye makeup has been found in Egyptian tombs dating to 3500 B.C. Today women of all ages around the world wear makeup to enhance their physical appearance.

It would be beneficial to have a cosmetic device that can be applied to the skin of a person's face and later removed without damage to the skin. Such a cosmetic device may be used to cover up an imperfect or damaged facial feature, or even to replace a missing facial feature.

### SUMMARY OF THE INVENTION

Several embodiments of a cosmetic device are disclosed. In general, the cosmetic device includes a thin, flexible substrate having two opposed sides, and an adhesive layer formed on one of the sides of the substrate. In some embodiments, a coloring layer is formed on the other side of the substrate. In another embodiment, multiple hair elements are distributed across, and have portions extending outwardly from, the other side of the substrate. Described cosmetic systems include multiple cosmetic devices releasably attached to release sheets. Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of one embodiment of a cosmetic device, wherein the cosmetic device is an eye shadow element;

FIG. 2 is a cross sectional view of one embodiment of the eye shadow element of FIG. 1 as indicated in FIG. 1;

FIG. 3 is an exploded perspective view of a cosmetic system including a second embodiment of the cosmetic device of FIG. 1 and a release sheet, wherein the cosmetic device includes a pair of lip-shaped elements;

FIG. 4 is a cross sectional view of the cosmetic system of FIG. 3 as indicated in FIG. 3, wherein the lip-shaped elements are releasably attached to the release sheet;

FIG. 5 is a perspective view of a third embodiment of the cosmetic device of FIG. 1, wherein the cosmetic device is a facial sheet adapted to be applied to a person's face;

FIG. 6 is a view of a cosmetic system including two copies of the facial sheet of FIG. 5 releasably mounted to a release sheet and a container containing a concealer;

FIG. 7 is a perspective view of another embodiment of the cosmetic device of FIG. 1, wherein the cosmetic device is an eyebrow element adapted to resemble a human eyebrow; and

FIG. 8 is a cross sectional view of one embodiment of the eyebrow element of FIG. 7 as indicated in FIG. 7.

### DETAILED DESCRIPTION OF THE INVENTION

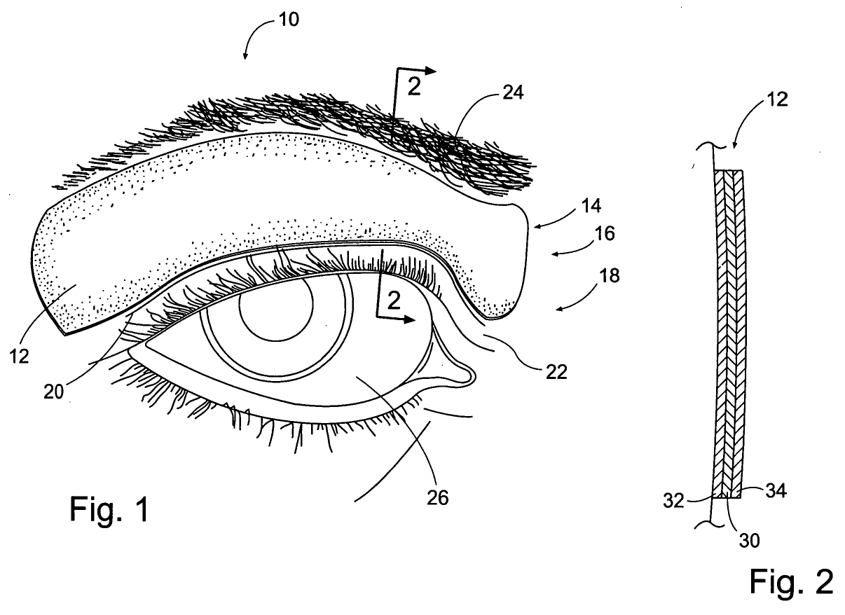

FIG. 1 is a perspective view of one embodiment of a cosmetic device 10, wherein the cosmetic device 10 is an eye shadow element 12. The eye shadow element 12 is shaped to fit over (i.e., substantially cover) an upper portion 14 of an upper eyelid 16 of a person's face 18, and to give the appearance of professionally applied eye shadow. In human eye surface anatomy, an upper eyelid crease 20 separates the upper portion 14 of the upper eyelid 16 from a lower portion 22. As shown in FIG. 1, the upper portion 14 of the upper eyelid 16 generally extends from the upper eyelid crease 20 to an eyebrow 24. When the person blinks, the lower portion 22 of the upper eyelid 16 spreads tears over an outer surface of an eyeball 26, while the upper portion 14 moves very little.

FIG. 2 is a cross sectional view of one embodiment of the eye shadow element 12 of FIG. 1 as indicated in FIG. 1. In the embodiment of FIG. 2, the eye shadow element 12 is a laminate structure including a thin film substrate 30 with an adhesive layer 32 formed on one side and a coloring layer 34 formed on an opposite side. In the embodiment of FIG. 2, the substrate 30 is formed from a thin, flexible material that is not readily visible on the skin. The substrate 30 may be formed from, for example, a thin plastic film material that is substantially transparent. Suitable plastic materials for the substrate 30 are believed to include polyethylene, polypropylene, polyvinyl chloride, and polyurethane.

The adhesive layer 32 is preferably a layer of a pressure sensitive adhesive material that adheres to skin and can be removed from the skin without damaging the skin. The adhesive material is preferably non-toxic and non-irritating. Suitable pressure sensitive adhesive material are well known in the medical and cosmetic arts, and are believed to include acrylic adhesives, rubber based adhesives, and styrene adhesives. The adhesive layer 32 may also be a layer of a water based and/or water washable adhesive material.

To reduce skin irritation, the substrate 30 preferably has many small holes passing therethrough (i.e., is preferably porous), and the adhesive layer 32 preferably conforms to the porous pattern of the substrate 30 such that the underlying skin can “breathe.” For example, the substrate 30 may be formed from a thin film material having many small holes (i.e., from a “microporous” thin film material).

In general, the coloring layer 34 includes coloring agent that simulates either a makeup color or a human skin color. The coloring agent may be, for example, a suitable paint or a stain. The coloring agent is preferably water resistant. The coloring layer 34 preferably has an outer surface that reflects light in substantially the same way that human skin reflects light. The coloring agent may be, for example, sprayed (e.g., airbrushed) onto the substrate 30.

The substrate 30 may be formed from a film material that already includes the adhesive layer 32. Alternately, the substrate 30 may be formed from a material that is inherently adhesive such that the adhesive layer 32 is unnecessary and may be eliminated. For example, GLAD® Press'n Seal™ sealable plastic wrap (The Glad Products Company, Oakland, Calif.) is a thin plastic film that either includes an adhesive layer or is inherently adhesive, and may be a suitable material from which to form the substrate 30.

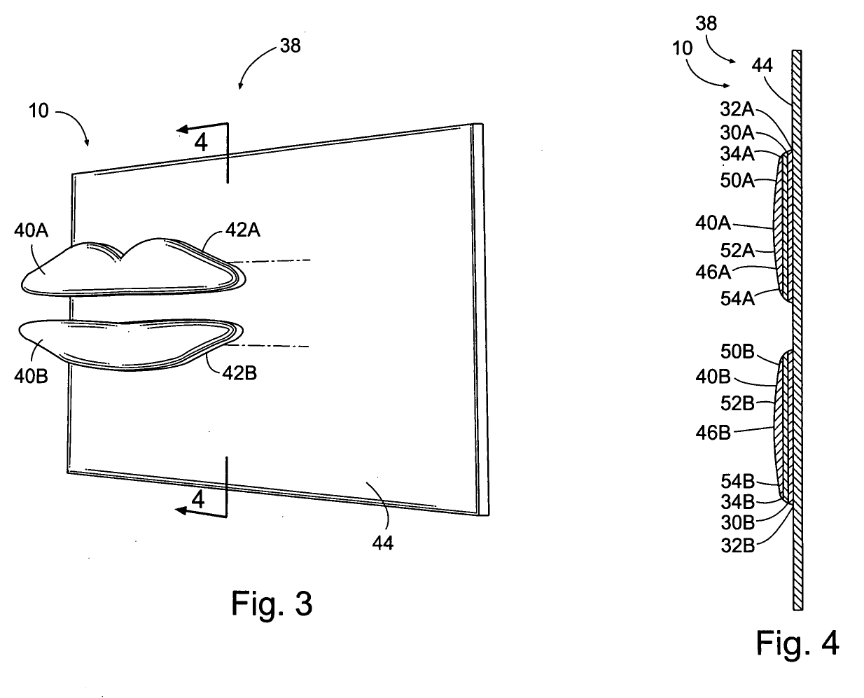

FIG. 3 is an exploded perspective view of a cosmetic system 38 including a second embodiment of the cosmetic device 10 of FIG. 1 and a release sheet 44, wherein the cosmetic device 10 includes a pair of lip-shaped elements 40A and 40B. In general, the lip-shaped elements 40A and 40B may be used to enhance a person's lips.

The lip-shaped elements 40A and 40B are shaped to fit over (i.e., substantially cover) a person's lips. More specifically, the lip-shaped element 40A has a perimeter 42A that is adapted to approximate the person's upper lip, and the lip-shaped element 40B has a perimeter 42B that is adapted to approximate the person's lower lip. Coloring layers of the lip-shaped elements 40A and 40B preferably include a coloring agent that simulates a lipstick color, but may also include a coloring agent that simulates a human skin color.

Adhesive layers of the lip-shaped elements 40A and 40B are adhered to the release sheet 44, and a user removes the lip-shaped elements 40A and 40B from the release sheet 44 prior to use. Several lip-shaped elements having different shapes may be attached to the release sheet 44 such that the user can select lip-shaped elements that most closely resembles shapes of the user's lips.

FIG. 4 is a cross sectional view of the cosmetic system 38 of FIG. 3 as indicated in FIG. 3, wherein the lip-shaped elements 40A and 40B are releasably attached to the release sheet 44. The lip-shaped elements 40A and 40B are mounted on the release sheet 44 via the adhesive layers so that they can be easily removed and applied. As illustrated in FIG. 4, the lip-shaped element 40A includes a body 46A. The body 46A includes a laminate structure having a thin film substrate 30A with an adhesive layer 32A formed on one side and a coloring layer 34A formed on an opposite side. The lip-shaped element 40B includes a body 46B. The body 46A includes a laminate structure having a thin film substrate 30B with an adhesive layer 32B formed on one side and a coloring layer 34B formed on the other side. The substrates 30A and 30B, the adhesive layers 32A and 32B, and the coloring layers 34A and 34B are similar to the respective substrate 30, adhesive layers 32, and coloring layer 34 of FIG. 2.

In general, the bodies 46A and 46B of the respective lip-shaped elements 40A and 40B may be formed from any suitable non-toxic materials. In a preferred embodiment, the bodies 46A and 46B also include a sugar or other sweet-tasting material.

In the embodiment of FIG. 4, the body 46A of the lip-shaped element 40A has a thicker center portion 52A and thinner top and bottom portions 50A and 54A, respectively, such that when the lip-shaped element 40A is attached to a person's lip, the person's lip appears full and plump. Similarly, the body 46B of the lip-shaped element 40B has a thicker center portion 52B and thinner respective top and bottom portions 50B and 54B for the same reason.

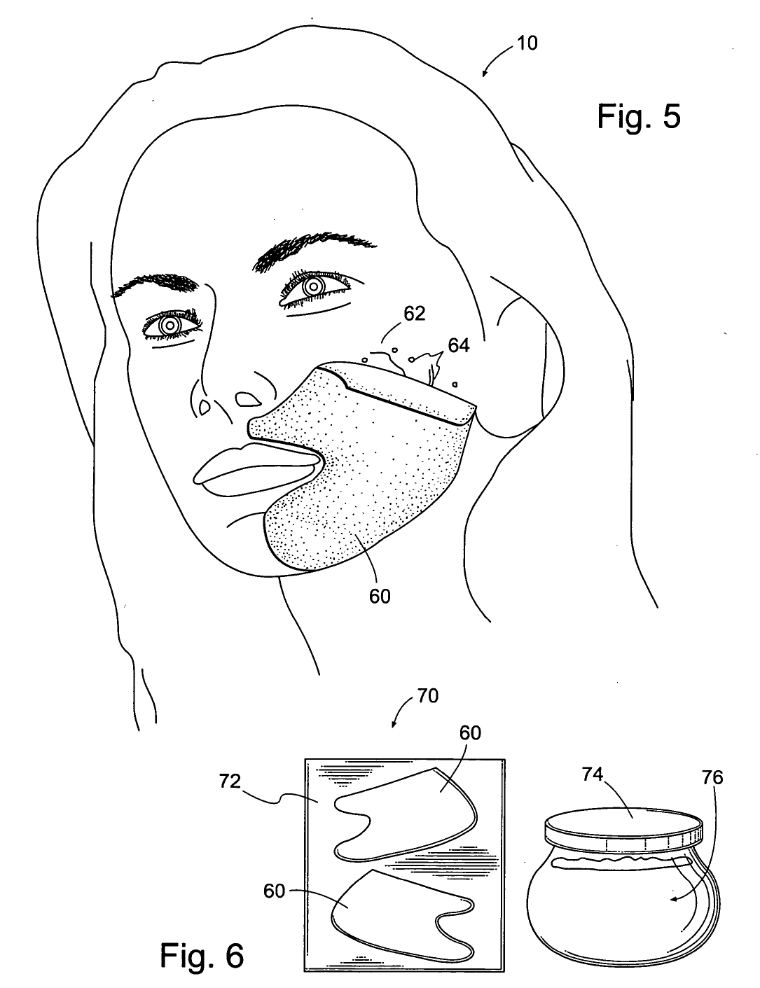

FIG. 5 is a perspective view of a third embodiment of the cosmetic device 10 of FIG. 1, wherein the cosmetic device 10 is a facial sheet 60 adapted to be applied to a person's face 62. The facial sheet 60 is preferably relatively thin, flexible, and adheres to the skin of the face 62. The facial sheet 60 is preferably thin enough to avoid being visually detectable, but also thick enough to cover imperfections 64 on the face 62 such as blemishes, acne scaring, etc. In general, the facial sheet 60 has a smooth outer surface that hides the existence of the imperfections 64.

The facial sheet 60 includes the laminate structure of FIG. 2 having the thin film substrate 30 with the adhesive layer 32 formed on one side and the coloring layer 34 formed on the opposite side. As described above, the coloring layer 34 includes a coloring agent to simulate either a makeup color or a human skin color. Further, the coloring layer 34 preferably has an outer surface that reflects light in substantially the same way that human skin reflects light.

FIG. 6 is a view of a cosmetic system 70 including two copies of the facial sheet 60 of FIG. 5 releasably mounted to a release sheet 72 and a container 74 containing a concealer 76. The two copies of the facial sheet 60 are mounted on the release sheet 72 via the adhesive layers so that they can be easily removed and applied.

In general, the concealer 76 is a facial cosmetic used to cover blemished skin. Suitable viscous liquid concealers are well known and widely available. The facial sheet 60 of FIGS. 5 and 6 is preferably used with the concealer 76. Once the facial sheet 60 has been applied to cover the imperfections 64 of FIG. 5, the concealer 76 is applied around the edges of and/or over the facial sheet 60 to hide the existence of the facial sheet 60.

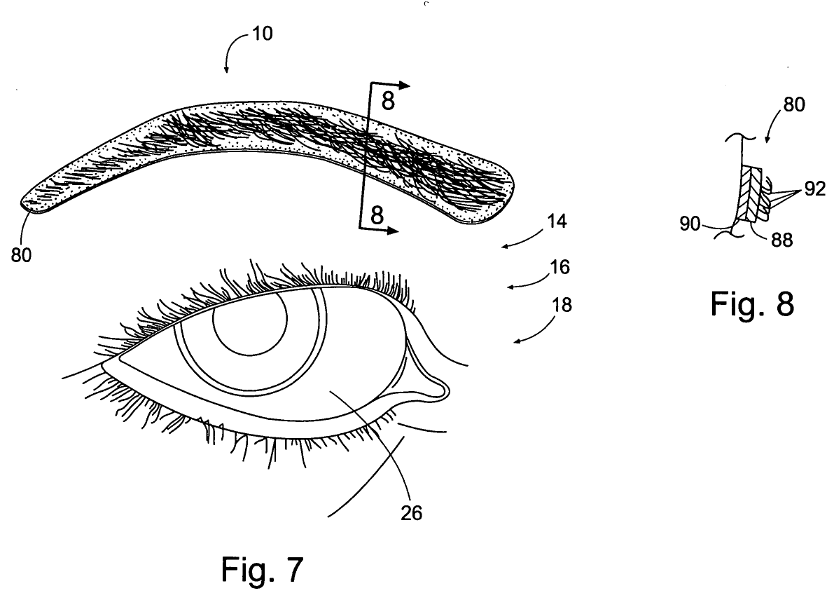

FIG. 7 is a perspective view of another embodiment of the cosmetic device 10 of FIG. 1, wherein the cosmetic device 10 is an eyebrow element 80 adapted to resemble a human eyebrow. The eyebrow element 80 may be used to replace a missing eyebrow, or to cover up an unattractive or imperfect eyebrow. In FIG. 7, objects shown in FIG. 1 and described above are numbered similarly. The eyebrow element 80 is expectedly positioned in the normal eyebrow position over a person's eye as shown in FIG. 7. More specifically, the eyebrow element 80 is expectedly positioned just above the upper portion 14 of the upper eyelid 16 of the person's face 18.

FIG. 8 is a cross sectional view of one embodiment of the eyebrow element 80 of FIG. 7 as indicated in FIG. 7. In the embodiment of FIG. 8, the eyebrow element 80 is a laminate structure including a substrate 88 with an adhesive layer 90 formed on one side. In general, the substrate 88 is formed from a thin, flexible material. The substrate 88 may be, for example, formed from a thin plastic film material. In general, the material used to form the substrate 88 is preferably not be readily visible on the skin. The material used to form the substrate 88 may be, for example, substantially transparent, or may include a coloring agent that simulates a makeup color or a human skin color. The substrate 88 preferably has an outer surface that reflects light in substantially the same way that human skin reflects light. Suitable plastic materials for the substrate 88 are believed to include polyethylene, polypropylene, polyvinyl chloride, and polyurethane. The adhesive layer 90 may be similar to the adhesive layer 32 shown in FIG. 2 and described above.

To reduce skin irritation, the substrate 88 preferably has many small holes passing therethrough (i.e., is preferably porous), and the adhesive layer 90 preferably conforms to the porous pattern of the substrate 30 such that the underlying skin can “breathe.” For example, the substrate 88 may be formed from a thin film material having many small holes (i.e., from a “microporous” thin film material).

In the embodiment of FIG. 8, multiple hair elements 92 are distributed across a surface of a side of the substrate 88 opposite the adhesive layer 90, and portions of the hair elements 92 extend outwardly from the side of the substrate 88 opposite the adhesive layer 90. In general, the hair elements 92 are adapted to resemble human eyebrow hairs. More specifically, the hair elements 92 preferably have lengths, thicknesses, and colors to simulate human eyebrow hairs, and are preferably curved like human eyebrow hairs.

In the embodiment of FIG. 8, each of the hair elements 92 has two opposed ends. One end of each of the hair elements 92 is anchored in the substrate 88, and the opposite end is free. In other embodiments both ends of the hair elements 92 may be anchored in the 88. Alternately, one or both ends of each of the hair elements 92 may be attached to the surface of the side of the substrate 88 opposite the adhesive layer 90.

It is noted that the adhesive layer of the cosmetic device 10 of FIGS. 1-8 allows the cosmetic device 10 to be attached to facial skin, and to be removed from the skin without damaging the skin. The different embodiments of the cosmetic device 10 may be used to give the appearance of professionally applied eye-shadow (FIGS. 1-2), to enhance a person's lips (FIGS. 3-4), to cover facial imperfections (FIGS. 5-6), or to replace a missing eyebrow or to cover up an unattractive or imperfect eyebrow (FIGS. 7-8).

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A cosmetic device, comprising:
a thin, flexible substrate having two opposed sides;
an adhesive layer formed on one of the sides of the substrate; and
a coloring layer formed on the other side of the substrate.

2. The cosmetic device as recited in claim 1, wherein the cosmetic device comprises an eye shadow element adapted to substantially cover an upper portion of an upper eyelid.

3. The cosmetic device as recited in claim 1, wherein the substrate is not readily visible when the cosmetic device is applied to the skin.

4. The cosmetic device as recited in claim 1, wherein the substrate is substantially transparent.

5. The cosmetic device as recited in claim 1, wherein the coloring layer has an outer surface that reflects light in substantially the same way that human skin reflects light.

6. The cosmetic device as recited in claim 1, wherein the adhesive layer comprises a layer of a pressure sensitive adhesive material.

7. The cosmetic device as recited in claim 1, wherein the adhesive layer comprises an adhesive material that is non-toxic and non-irritating.

8. The cosmetic device as recited in claim 1, wherein the substrate is porous, and wherein the adhesive layer conforms to a porous pattern of the substrate.

9. The cosmetic device as recited in claim 1, wherein the coloring layer comprises a coloring agent that simulates either a makeup color or a human skin color.

10. The cosmetic device as recited in claim 9, wherein the coloring agent comprises a paint or a stain.

11. The cosmetic device as recited in claim 9, wherein the coloring agent is water resistant.

12. The cosmetic device as recited in claim 1, wherein the cosmetic device comprises a lip-shaped element adapted to substantially cover a lip of a user.

13. The cosmetic device as recited in claim 1, wherein the coloring layer comprises a coloring agent that simulates a lipstick color.

12. The cosmetic device as recited in claim 1, wherein the cosmetic device comprises a facial sheet adapted to be applied to a face of a user.

13. A cosmetic system, comprising:
a plurality of cosmetic devices, each comprising:
a thin, flexible substrate having two opposed sides;
an adhesive layer formed on one of the sides of the substrate;
a coloring layer formed on the other side of the substrate;
a release sheet; and
wherein the cosmetic devices are releasably attached to the release sheet.

14. The cosmetic system as recited in claim 13, wherein the cosmetic devices comprise lip-shaped elements each adapted to substantially cover a lip of a user.

15. The cosmetic device as recited in claim 13, wherein the coloring layer comprises a coloring agent that simulates a lipstick color.

16. The cosmetic system as recited in claim 13, wherein the cosmetic devices are mounted on the release sheet via the adhesive layer.

17. The cosmetic system as recited in claim 13, wherein the cosmetic devices comprises facial sheets each adapted to be applied to a person's face.

18. A cosmetic device, comprising:
a thin, flexible substrate having two opposed sides;
an adhesive layer formed on one of the sides of the substrate; and
a plurality of hair elements distributed across, and having portions extending outwardly from, the other side of the substrate.

19. The cosmetic device as recited in claim 18, wherein the cosmetic device is adapted to resemble a human eyebrow.

20. The cosmetic device as recited in claim 18, wherein the substrate comprises a coloring agent that simulates either a makeup color or a human skin color.
